(19) United States
(12) Patent Application Publication
(10) Pub. No.: US 2010/0129267 A1
(43) Pub. Date: May 27, 2010

(54) AIR CLEANING DEVICE

(75) Inventors: NAKAO AKUTSU, Chu-Nan (TW); CHIH-MING LAI, Chu-Nan (TW)

Correspondence Address: PCE INDUSTRY, INC.;ATT. Steven Reiss, 288 SOUTH MAYO AVENUE, CITY OF INDUSTRY, CA, 91789, US

(73) Assignee: FOXSEMICON INTEGRATED TECHNOLOGY, INC., Chu-Nan (TW)

(21) Appl. No.: 12/497,643

(22) Filed: Jul. 3, 2009

(30) Foreign Application Priority Data
Nov. 27, 2008 (CN) 200810305778.8

(51) Int. Cl. A61L 9/20

(52) U.S. Cl. 422/121

(57) ABSTRACT

An exemplary air cleaning device includes a roller, a photo-catalyst layer and a light source. The roller includes a base and a plurality of fins installed on the base. The roller is movable to cause an air to flow. The photo-catalyst layer is formed on the fins. The light source is disposed facing toward the roller for irradiating the photo-catalyst layer to cause a photocatalytic reaction thus cleaning the air flowing therethrough.

### BACKGROUND

1. Technical Field

The present disclosure generally relates to an air cleaning device.

2. Discussion of Related Art

Air pollution has long been recognized as a threat to human health as well as to earth's ecosystems. In such, air cleaning devices are widely used to improve air quality of the environment.

Most conventional air cleaning devices use activated carbon or ozone to filter and absorb contaminants and sterilize microbes in the air. Ozone has strong oxidative function, it can oxidize organic pollutant in the air immediately and decomposed into vitriol and nitric acid. But vitriol and nitric acid are also harmful to the human health and the environment. Also, activated carbon may loose its effect (e.g. absorbing efficiency) if it is exposure to air for a long time.

What is needed, therefore, is an improved air cleaning device which can overcome the above shortcomings.

### BRIEF DESCRIPTION OF THE DRAWINGS

Many aspects of the present air cleaning device can be better understood with reference to the following drawings. The components in the drawings are not necessarily drawn to scale, the emphasis instead being placed upon clearly illustrating the principles of the present air cleaning device. Moreover, in the drawings, like reference numerals designate corresponding parts throughout certain views.

FIG. 1 is a schematic view of air cleaning device, according to a first exemplary embodiment.

FIG. 2 is a schematic enlarged view of a circled portion of FIG. 1.

FIG. 3 is a schematic view of air cleaning device, according to a second exemplary embodiment.

### DETAILED DESCRIPTION OF EMBODIMENTS

Reference will now be made to the drawings to describe embodiments of the present air cleaning device, in detail.

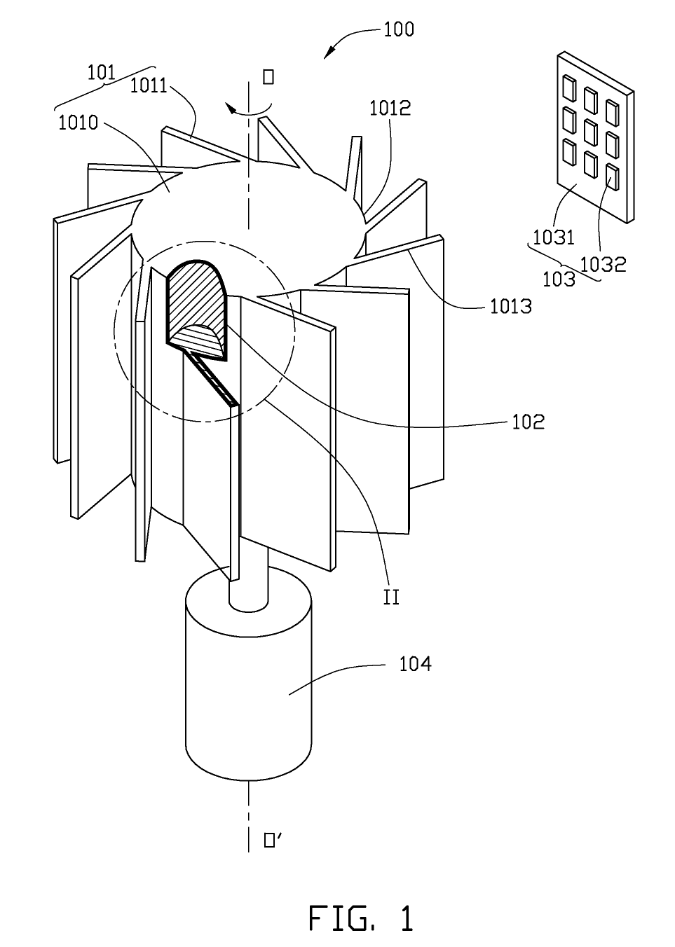

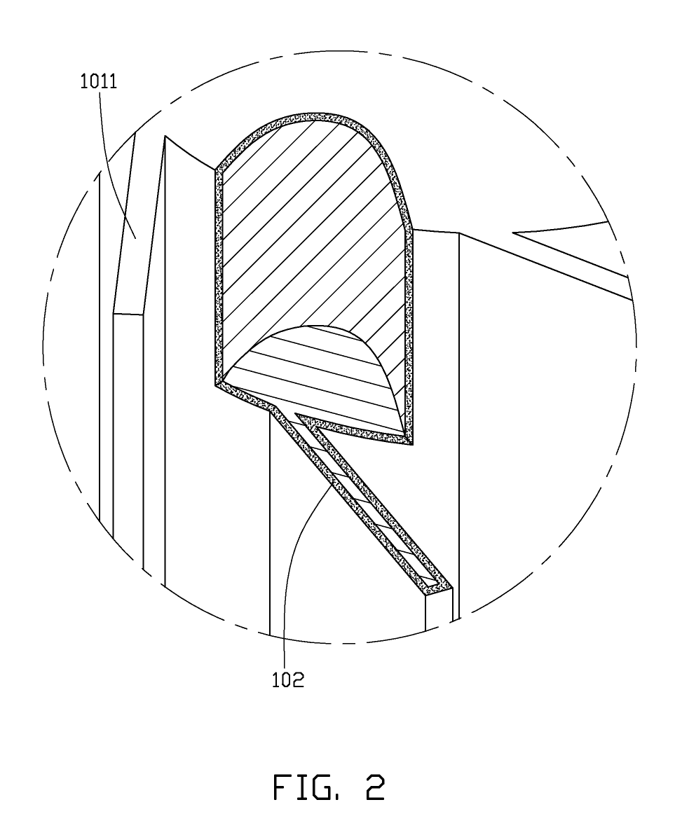

Referring to FIGS. 1 and 2, an air cleaning device 100, according to a first embodiment, includes a roller 101, a photo-catalyst layer 102 and a light source 103.

The roller 101 comprising a columnar base 1010 and a plurality of fins 1011 installed on the base 1010. The base 1010 has a central axis OO′. The fins 1011 extend outwards from the roller 101 and are inclined in a direction opposite to the rotational direction of the roller 101. In the present embodiment, each of the fins 1011 extend at an acute angle measured clockwise from an imaginary line tangential to the circumference of the roller 101. In an exemplary embodiment, the base 1010 is integrally formed with the fins 1011. Each of the fins 1011 has an external surface 1013, and the base 1010 has a discontinuous external surface 1012 between every two adjacent fins 1011. The roller 101 is capable of rotating manually or automatically and the fins 1011 rotate with the roller 101 to create an airflow. The photo-catalyst layer 102 is formed on the external surface 1012 of the base 1010 and the external surfaces 1013 of the fins 1011. The photo-catalyst layer 102 contains titanium dioxide (TiO₂), tin oxide (SnO₂), zinc oxide (ZnO), tungsten oxide (WO₃), iron oxide (Fe₂O₃), SeTiO₃, cadmium selenide (CdSe), KTaO₃, cadmium sulfide (CdS) or niobium oxide (Nb₂O₅). Generally, the photo-catalyst layer 102 contains a material selected from the group consisting of chromium, vanadium, carbon, nitrogen and sulfur and any combination thereof. When airflow is generated, the photo-catalyst layer 102 can initiate photocatalytic reactions with organic contaminants contained in the air, excited by the light from the light source 103. Therefore, the organic contaminants can be oxidized and decomposed into carbon dioxide (CO₂) and water (H₂O). It can be understood that the photo-catalyst layer 102 may be only formed on the external surfaces 1013 of the fins 1011.

The light source 103 comprising a substrate 1031 and a plurality of light emitting elements 1032. The light emitting elements 1032 are arranged on the substrate 1031 and facing toward external surfaces 1013 of the fins 1011, therefore, light emitted from the light emitting elements 1032 can irradiate the photo-catalyst layer 102. The light emitting elements 1032 are selected from the group consisting of a light emitting diode chip, a light emitting diode and a light emitting diode module. In the present embodiment, the light emitting elements 1032 are white light emitting diodes and can be used for illuminating.

The air cleaning device 100 further comprises a drive module 104. The drive module 104 is mechanically connected with the base 1010 of the roller 101. The drive module 104 is configured for driving the roller 101 rotates around the central axis OO′. In general, the drive module 104 is a motor.

In the present embodiment, the drive module 104 drives the roller 101 rotate and creates airflow with rotating of the fins 1011. Thus, an inlet and an outlet are not needed. Additionally, the photo-catalyst layer 102 is formed on the external surfaces 1013 of the fins 1011, the area of the photo-catalyst layer 102 is enlarged, therefore, the efficiency of the air cleaning device 100 is improved.

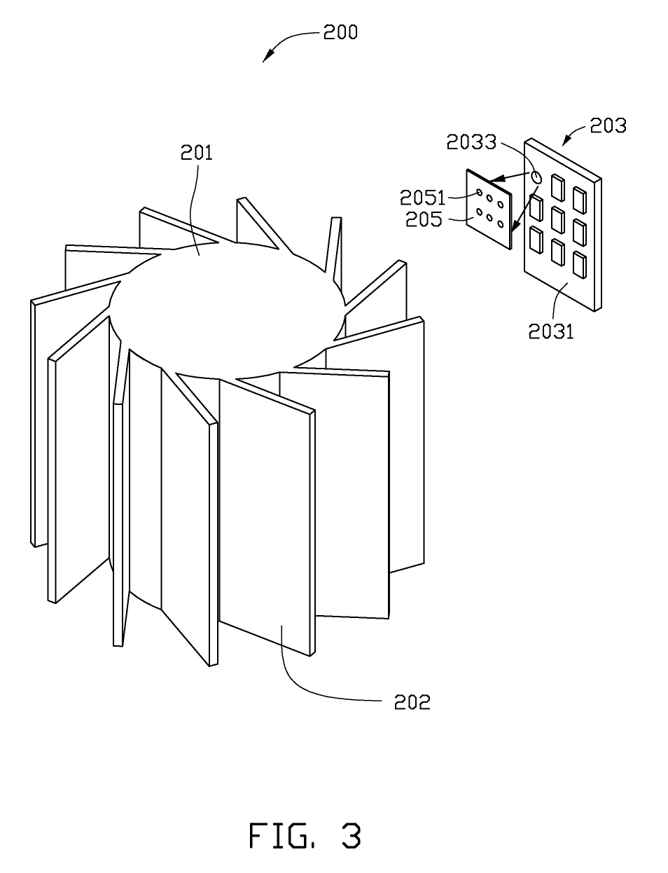

Referring to FIG. 3, an air cleaning device 200, in accordance with a second embodiment. The air cleaning device 200 is similar to the air cleaning device 100 except that an ultraviolet (UV) light source 2033 and a light pervious board 205 are included.

The light pervious board 205 is arranged between a light source 203 and a roller 201. A plurality of through holes 2051 are formed in the light pervious board 205. The light pervious board 205 is doped with a phosphors layer thereon. The material of phosphors can absorb UV light and change the wavelength of UV light to visual light.

The light source 203 includes a substrate 2031 and an UV light source 2033. The UV light source 2033 is arranged on the substrate 2031 and opposite to the light pervious board 205. Preferably, the UV light source 2033 is a UV light emitting diode. Additionally, there can be more than one UV light source 2033 arranged on the substrate 2031.

A part of UV light form the UV light source 2033 can pass the through holes 2051 to irradiate the photo-catalyst layer 202, and the photo-catalyst layer 202 initiate photocatalytic reactions with organic contaminants adsorbed in the air and decomposed into carbon dioxide (CO₂) and water (H₂O).

Other part of UV light form the UV light source 2033 irradiate to the light pervious board 205, and the phosphors in the light pervious board 205 can absorb UV light and change the wavelength of UV light to visual light. The visual light not only can be used to excite the photo-catalyst layer 202 but also can be used to illuminate.

Preferably, the light pervious board 205 is movable relative to the light source 203 by manually or automatically. Therefore, when the air cleaning device 200 is only used for cleaning air, the light pervious board 205 is moved away from the light path of the UV light source 2033, so that all UV light emitted from the UV light source 2033 can irradiate the photo-catalysis layer 202 and improve efficiency of the air cleaning device 200.

The photocatalytic reactions of the photo-catalyst layer 202 with the UV light is much faster than that of the visual light, therefore, the efficiency of the air cleaning device 200 is improved. Additionally, the air cleaning device 200 includes a light pervious board 205 which has phosphor therein, the phosphor can absorb part of the UV light to limit excess UV light that is harmful to human health.

Advantageously, the air cleaning device can be used for illumination.

It is to be understood, however, that even though numerous characteristics and advantages have been set forth in the foregoing description of embodiments, together with details of the structures and functions of the embodiments, the disclosure is illustrative only, and changes may be made in detail, especially in matters of shape, size, and arrangement of parts within the principles of the disclosure to the full extent indicated by the broad general meaning of the terms in which the appended claims are expressed.

What is claimed is:

1. An air cleaning device, comprising:
a roller comprising a base and a plurality of fins installed on the base, the roller being rotatable to cause an air to flow;
a photo-catalyst layer being formed on the fins;
a light source being disposed facing toward the roller for irradiating the photo-catalyst layer to cause a photocatalytic reaction thus cleaning the air flowing therethrough.

2. The air cleaning device of claim 1, further comprising a drive module, the drive module is mechanically connected with the base of the roller and configured for driving the roller to rotate.

3. The air cleaning device of claim 1, wherein the photo-catalyst layer contains TiO₂.

4. The air cleaning device of claim 1, wherein the photo-catalyst layer contains a material selected from the group consisting of chromium, vanadium, carbon, nitrogen and sulfur.

5. The air cleaning device of claim 1, wherein the light source is selected from the group consisting of a light emitting diode chip, a light emitting diode and a light emitting diode module.

6. The air cleaning device of claim 1, wherein the light source includes at least one ultraviolet light source.

7. The air cleaning device of claim 6, wherein the ultraviolet light source is an ultraviolet light emitting diode.

8. The air cleaning device of claim 7, further comprising a light pervious board with a phosphor layer thereon, the light pervious board is arranged between the ultraviolet light emitting diode and the roller.

9. The air cleaning device of claim 8, wherein a plurality of through holes are formed in the light pervious board.

10. The air cleaning device of claim 8, wherein the light pervious board is movable relative to the light source.

11. The air cleaning device of claim 1, wherein a photo-catalyst layer is formed on the base of the roller.

12. The air cleaning device of claim 1, wherein the fins extend outwards from the roller, and are inclined in a direction opposite to the rotational direction of the roller.
